(19) United States
(12) Patent Application Publication
(10) Pub. No.: US 2022/0130039 A1
(43) Pub. Date: Apr. 28, 2022

(54) SYSTEM AND METHOD FOR TUMOR TRACKING

(71) Applicant: Surgical Theater, Inc., Los Angeles, CA (US)

(72) Inventors: Mordechai Avisar, Highland Heights, OH (US); Alon Yakob Geri, Orange Village, OH (US); Gidon Navrotsky, Binyamina (IL)

(73) Assignee: SURGICAL THEATER, INC., Los Angeles, CA (US)

(21) Appl. No.: 17/510,260

(22) Filed: Oct. 25, 2021

Related U.S. Application Data
(60) Provisional application No. 63/105,089, filed on Oct. 23, 2020.

(51) Int. Cl. G06T 7/00; G16H 10/60; G16H 30/20; G06F 3/01; A61B 34/00; A61B 34/10

(52) U.S. Cl. CPC G06T 7/0012; G16H 10/60; G16H 30/20; G06F 3/011; A61B 2034/256; A61B 34/10; G06T 2207/30096; G06T 2207/20092; A61B 34/25

(57) ABSTRACT

A tumor tracking system and method which leverages an image generator and an MD6DM model for tracking and monitoring a status of a tumor over time. In particular, the tumor tracking system enables a physician, or a group of physicians, in collaboration, to visualize and interact with an integrated representation of data relating to a patient's tumor and associated treatment history and also to plan future treatments of the tumor. An integrated and interactive tumor board generated by the tumor tracking system provides a single interface that compiles and organizes information from multiple sources to enable efficient tumor tracking and treatment planning.

### CROSS-REFERENCES TO RELATED APPLICATIONS

This application claims the benefit of U.S. provisional patent application Ser. No. 63/105,089, filed on Oct. 23, 2020, incorporated herein by reference.

### BACKGROUND

Treating a patient for a tumor can be a complex endeavor which may span over an extended time period. For example, treatments may include surgical procedures, radiation, or chemotherapy, and may occur over the course of several months or years. Moreover, a physician may perform multiple image scans, such as CT or MRI, over time to visualize the changes in the tumor as different treatments are administered. The physician may need to monitor and compare results of past treatments performed by reviewing historical scans and images in order to decide on future treatments based on the size and location of the tumor and the change in size over time. In addition, it may be desirable for multiple physicians to collaborate. For example, a group of physicians may meet regularly or occasionally to collaborate and review a patient's case in order to decide on next steps and future treatment. However, compiling, organizing, and reviewing this information in a collaborative manner may be tedious, inefficient, time consuming, and prone to error.

### SUMMARY

Provided are a plurality of example embodiments, including, but not limited to, a method for tracking a tumor, comprising:
a tumor tracking computer receiving multiple medical images of a particular patient, representative of the patient's tumor over time;
the tumor tracking computer receiving multiple interactive models of the patient generated using the medical images of the patient, said interactive models being representative of the patient's tumor over time;
the tumor tracking computer receiving an electronic health record of the patient;
the tumor tracking computer generating a multi-dimensional interactive virtual reality view of the tumor in real-time utilizing the multiple interactive models and the multiple medical images;
the tumor tracking computer generating and displaying a synchronized and integrated tumor board including a timeline of the tumor and a viewer displaying the multi-dimensional interactive virtual reality view; and
the tumor tracking computer receiving one or more inputs from a user to navigate the timeline to update the multi-dimensional interactive virtual reality view over time.
Further provided is a method of tracking a tumor, comprising:
a tumor tracking computer receiving multiple medical images of a particular patient, representative of the patient's tumor over time;
the tumor tracking computer receiving multiple interactive models of the patient generated using the medical images of the patient, said interactive models being representative of the patient's tumor over time;
the tumor tracking computer receiving an electronic health record of the patient;
the tumor tracking computer generating a multi-dimensional interactive virtual reality view of the tumor in real-time utilizing the multiple interactive models and the multiple medical images;
the tumor tracking computer generating and displaying a synchronized and integrated tumor board, said tumor board including:
   displaying a timeline of the tumor including a history of treatment(s) and/or surgery used to treat the tumor,
   displaying the electronic health records,
   displaying at least the one of said medical images, and
   displaying the multi-dimensional interactive virtual reality view; and
the tumor tracking computer receiving one or more inputs from a user to navigate the timeline to update in real-time the display of the medical images viewer and the interactive model viewer over time.

Also provided are additional example embodiments, some, but not all of which, are described hereinbelow in more detail.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example system for tracking a tumor;

FIG. 2 illustrates an example tumor board for tracking a tumor;

FIG. 3 illustrates an example tumor board for tracking a tumor;

FIG. 4 illustrates an example tumor board for tracking a tumor;

FIG. 5 illustrates an example tumor board for tracking a tumor;

FIG. 6 illustrates an example tumor board for tracking a tumor;

FIG. 7 illustrates an example tumor board for tracking a tumor;

FIG. 8 illustrates an example tumor board for tracking a tumor;

FIG. 9 is a flow chart of an example method for tracking a tumor; and

FIG. 10 is a block diagram of an example computer for implementing an example tumor tracking computer of FIG. 1.

### DETAILED DESCRIPTION

The following acronyms and definitions will aid in understanding the detailed description:

VR—Virtual Reality—A 3Dimensional computer generated environment which can be explored and interacted with by a person in varying degrees.

HMD—Head Mounted Display refers to a headset which can be used in VR environments. It may be wired or wireless. It may also include one or more add-ons such as headphones, microphone, HD camera, infrared camera, hand trackers, positional trackers etc.

SNAP Model—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

Fly-Through—Also referred to as a tour, this describes a perspective view of a virtual reality environment while moving through the virtual reality environment along a defined path.

A surgery rehearsal and preparation tool previously described in U.S. Pat. No. 8,311,791, incorporated in this application by reference, has been developed to convert static CT and MM medical images into dynamic and interactive multi-dimensional full spherical virtual reality, six (6) degrees of freedom models (“MD6DM”) based on a prebuilt SNAP model that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional two-dimensional patient medical scans, that gives spherical virtual reality 6 degrees of freedom (i.e. linear; x, y, z, and angular, yaw, pitch, roll) in the entire volumetric spherical virtual reality model.

The MD6DM is rendered in real time by an image generator using a SNAP model built from the patient's own data set of medical images including CT, MM, DTI etc., and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved and can be appreciated using the MD6DM.

The algorithm of the MD6DM rendered by the image generator takes the medical image information and builds it into a spherical model, a complete continuous real time model that can be viewed from any angle while “flying” inside the anatomical structure. In particular, after the CT, Mill, etc. takes a real organism and deconstructs it into hundreds of thin slices built from thousands of points, the MD6DM reverts it to a 3D model by representing a 360° view of each of those points from both the inside and outside.

Described herein is a tumor tracking system, leveraging an image generator and a MD6DM model, for tracking and monitoring a status of a tumor over time. In particular, the tumor tracking system enables a physician, or a group of physician in collaboration, to visualize and interact with an integrated representation of data relating to a patient's tumor and associated treatment history and also to plan future treatments of the tumor. An integrated and interactive tumor board generated by the tumor tracking system provides a single interface that compiles and organizes information from multiple sources to enable efficient tumor tracking and treatment planning.

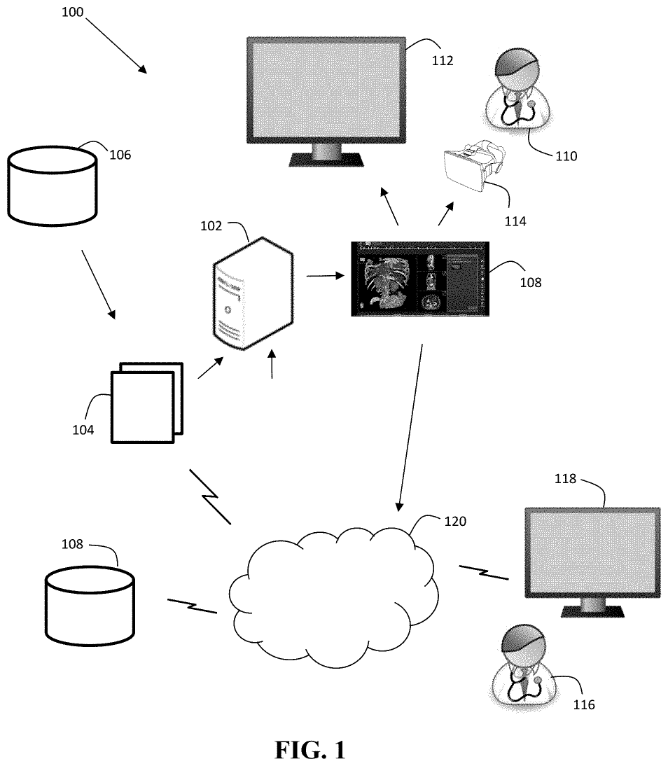

FIG. 1 illustrates an example tumor tracking system 100 for tracking and monitoring a status of a tumor over time. The tumor tracking system 100 includes a tumor tracking computer 102 for receiving patient tumor data 104 from multiple data sources. Patient tumor data 104 may include any suitable data for monitoring, tracking and treating a tumor. For example, patient tumor data 104 may include one or more medical images such as CT scans and Mills. Patient tumor data 104 may further include information regarding the treatments that have already been administered to the patient, any associated medical notes, and other relevant patient data. Patient tumor data 104 may further include patient-specific SNAP models.

In one example, the tumor tracking computer 102 receives patient tumor data 104 from a local data source 106 located proximate to or integrated with the tumor tracking computer 102. In another example, the tumor tracking computer 102 receives patient tumor data 104 from a network data source 108 via a network 120 such an enterprise network or the Internet. In one example, the network data source 108 may be a third-party data source such as an EMR provider.

The tumor tracking computer 102 integrates the patient tumor data 104 into a tumor board 108 for physician 110 interaction. The tumor tracking computer 102 further includes an image generator (not shown) for rendering a MD6DM using a received SNAP model. In one example, the tumor tracking computer 102 also synchronizes the patient tumor data 104 such that as the physician 110 interacts with a first portion of the patient tumor data 104 via the tumor board 108, the remaining portion of the patient tumor data 104 is updated and presented via the tumor board 108 accordingly. In one example, the tumor board 108 provides for a read-only experience in which the physician 110 may only visualize the patient tumor data 104 without making any changes to the data. In another example, the tumor board 108 provides an experience in which the physician 110 may make changes to the patient tumor data 104, such as updating a patient health record, adding a note, recommending a treatment plan, etc.

The tumor tracking computer 102 presents the tumor board 108 to the physician 110 on a display 112 for viewing and interaction via suitable user input mechanisms. In another example, tumor tracking computer 102 may present the tumor board 108 to the physician 110 via a HMD 114 for a more immersive experience. In one example, tumor tracking computer 102 may present the tumor board 108 to a remote physician 116 via a remote display 118 over the network 120 to enable collaboration. In such an example, the tumor tracking computer 102 may present the identical tumor board 108, including any interactions with the tumor board 108 by any number of multiple physicians, simultaneously to the multiple physicians such that the multiple physicians are all experiencing the same view for efficient collaboration.

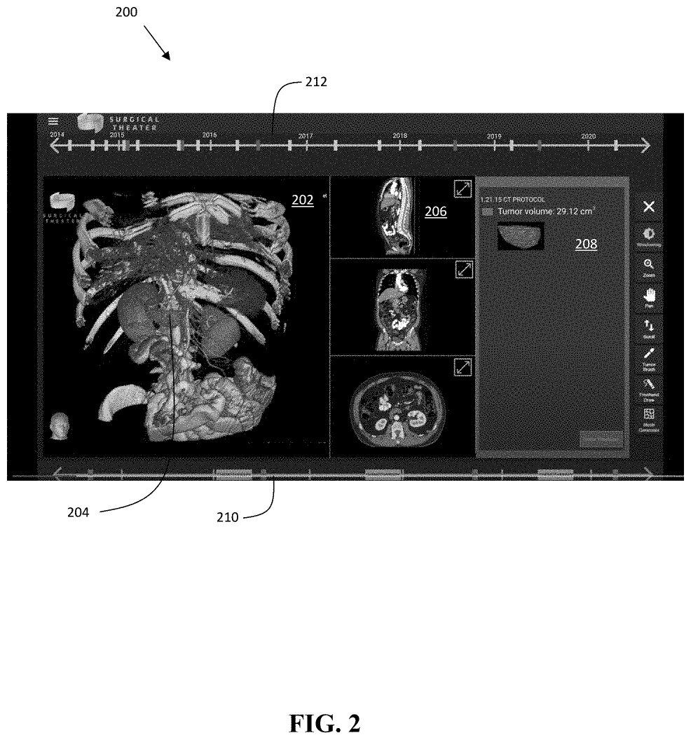

FIG. 2 illustrates an example tumor board 200. The tumor board 200 includes a MD6DM viewer 202 for visualizing a MD6DM rendered in real time by an image generator using a SNAP model. The MD6DM viewer 202 enables fully interactive 360 navigation and exploration of an inside of the patient's anatomy in order to view and examine a tumor 204. The tumor board 200 further includes a DICOM viewer 206 for viewing and interacting with DICOM images such as Mill and CT scans that correspond to the MD6DM in the MD6DM viewer 202. In one example, the DICOM viewer 206 and the MD6DM viewer 202 are synchronized such that an interaction or a change in perspective of view of the anatomy in one viewer automatically causes the other viewer to update and display the same perspective of view of the anatomy. Tumor board 200 further includes a tumor viewer 208 which provides a rendering of the tumor along with relevant data such as the tumor size.

In order to facilitate the review and analysis of the tumor 204 over time, the tumor board 200 includes a timeline for navigating historical data relating to the tumor 204. In one example, the timeline may be divided into multiple components. For example, as illustrated, the tumor board 200 includes a treatment timeline 210 and an imaging and surgery timeline 212. It should be appreciated that the number of timelines may be expanded or combined as suitable to appropriately provide a physician with an effective interactive experience for visualizing the data.

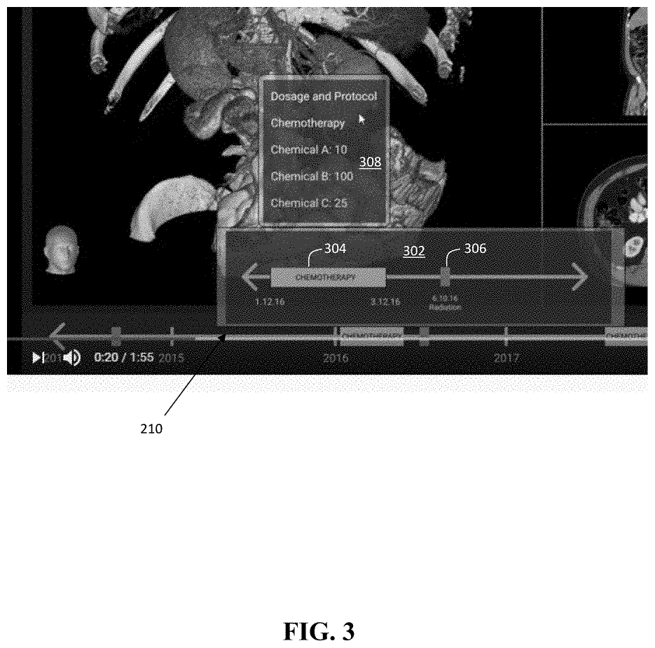

As further illustrated in FIG. 3, selecting a point on the treatment timeline 210 opens an expanded treatment timeline view 302 of the timeline for a predetermined date range based on where on the treatment timeline 210 a selection was made. The expanded treatment timeline view 302 provides a more detailed view of the different treatments that have been administered to a patient on given dates. For example, a chemotherapy treatment may be represented on by a chemotherapy icon 304 having a first type of appearance and a radiation treatment may be represented by a radiation icon 306 having a second type of appearance, different from that of the chemotherapy icon 304. Thus, a physician may be able to easily view a history of treatments and distinguish between the different types of treatments by examining the treatment timeline 210 and opening an expanded treatment timeline view 302 as needed. Selecting a treatment icon on either the treatment timeline view 210 or within the expanded treatment timeline view 302 causes a detailed treatment view 308 to display specific information related to a particular treatment. For example, selecting a chemotherapy treatment may result in information such as the type of drugs used and the dosage administered to be displayed in the detailed treatment view 308.

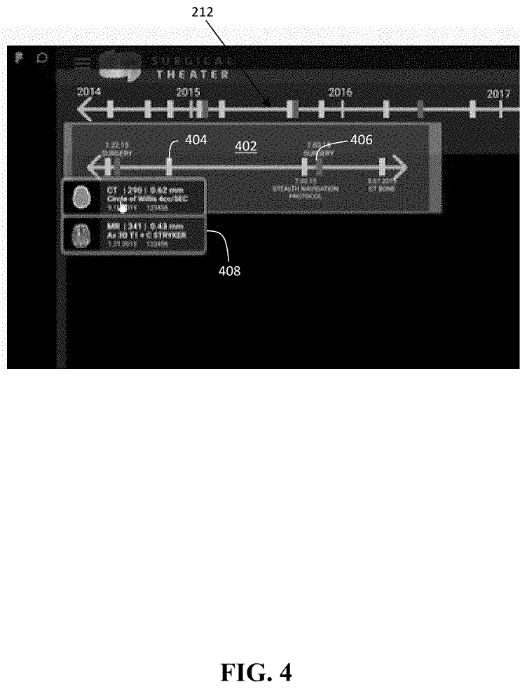

Similarly, as illustrated in FIG. 4, selecting a point on the imaging and surgery timeline 212 opens an expanded imaging timeline view 402 of the timeline for a predetermined date range based on where on the imaging timeline 212 a selection was made. The expanded imaging timeline view 402 provides a more detailed view of the different medical images that have been taken for a patient on given dates as well as the different surgical procedures that have performed on the patient on given dates. For example, a medical image taken may be represented on by an image icon 404 having a first type of appearance and a performed surgery may be represented by a surgery icon 406 having a second type of appearance, different from that of the chemotherapy icon 404. Selecting an icon on either the imaging timeline 212 or within the expanded imaging timeline view 402 causes a detailed view to display more specific information. For example, selecting a surgery icon 406 may result in more detailed information about a specific surgery to be displayed in a detailed surgery view (not shown). Similarly, selecting an image icon 404 may result in a more detailed information about the types of medical images taken to be displayed in a detailed medical imaging view 408.

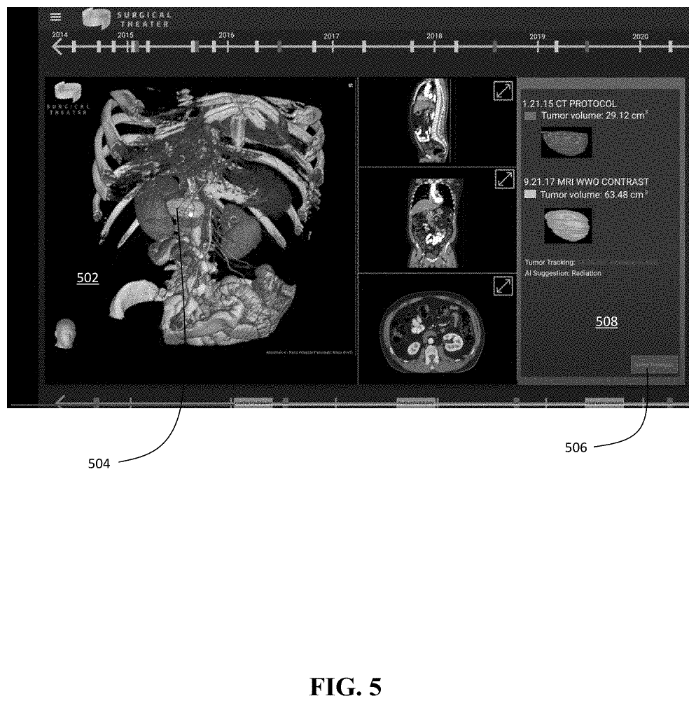

Selecting a medical image from the detailed medical imaging view 408 causes the MD6DM viewer 502, as illustrated in FIG. 5, to render an updated view of the MD6DM, including an updated view of the tumor 504. In one example, the tumor 504 is a representation of the tumor over multiple instances in time, rendered in two or more different colors, each color representing a change in the tumor's 504 size and shape over time. This is further illustrated by the tumor viewer 508 which renders separate views of the tumor 504 in different colors, each color representing the shape and size of the tumor at a specific time. Thus, be selecting different medical scans from the imaging and surgery timeline 212, a physician is easily able to compare the size of the tumor over time and asses the effectiveness of the different treatments and surgeries administered over time based on the change in the tumor. In circumstances where DICOM images have been taken of a patient before and after a surgical procedure, the physician may be able to easily and efficiently flip back and forth between different images and models, pre-surgery and post surgery to analyze the effectiveness of a particular surgical procedure.

In one example, the tumor tracking computer 102 has artificial intelligence capabilities and is able to make recommendations, via the tumor viewer 508 in the tumor board, for how to treat a patient's tumor. For example, the tumor tracking computer 102 may learn from historical tumor data and develop its own algorithms on what combinations of radiation, chemotherapies, and surgeries are most effective for treating certain types of tumors. The tumor tracking computer 102 may asses the current tumor being tracked and evaluated and make a treatment recommendation based on its own AI algorithms.

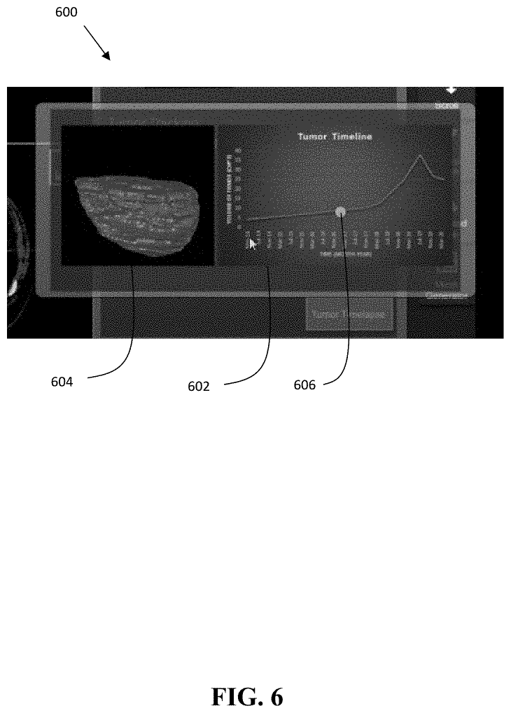

The tumor viewer 508 includes a tumor timeline feature 506, which upon selection, generates a tumor timeline window 600 as illustrated in FIG. 6. The tumor timeline window 600 includes a graphical representation window 602 which illustrates in graphical form the change in size of the tumor over time. In one example, if tumor data is limited, interpretations or extrapolations may be made in order to create the graphical representation. The tumor timeline window 600 further includes an animated tumor window 604 which shows an animation of the tumor changing shape and size over time. In one example, a graphical marker 606 moves along the graph in the graphical representation window 602 to the corresponding point in time as the animation of the tumor in the animated tumor window 604 changes according to the same timeline.

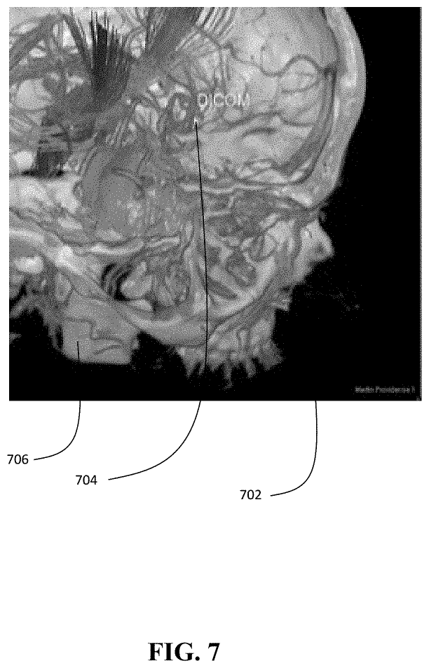

In one example, as illustrated in FIG. 7, the MD6DM image viewer 702 displays a moveable DICOM marker 704 which can be positioned anywhere on the MD6DM 706. As the DICOM marker 704 is moved, a corresponding marker (not shown) is also displayed on the DICOM in the DICOM viewer so that a physician may more easily associate the DICOM with MD6DM and visualize both simultaneously and seamlessly.

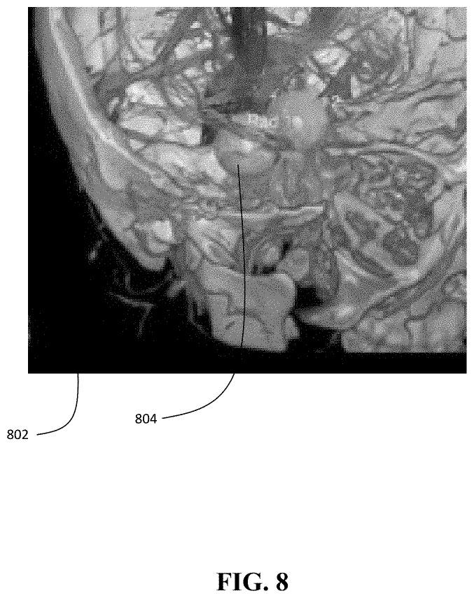

In one example, as illustrated in FIG. 8, the MD6DM image viewer 802 integrates a radiation heat map marker 804 indicating a plan for performing radiation on the tumor. By integrating the heat map marker 804, a physician is able to visualize and focus on an area requiring surgery and formulate a plan for removing the portion of the tumor that a radiation treatment will not remove.

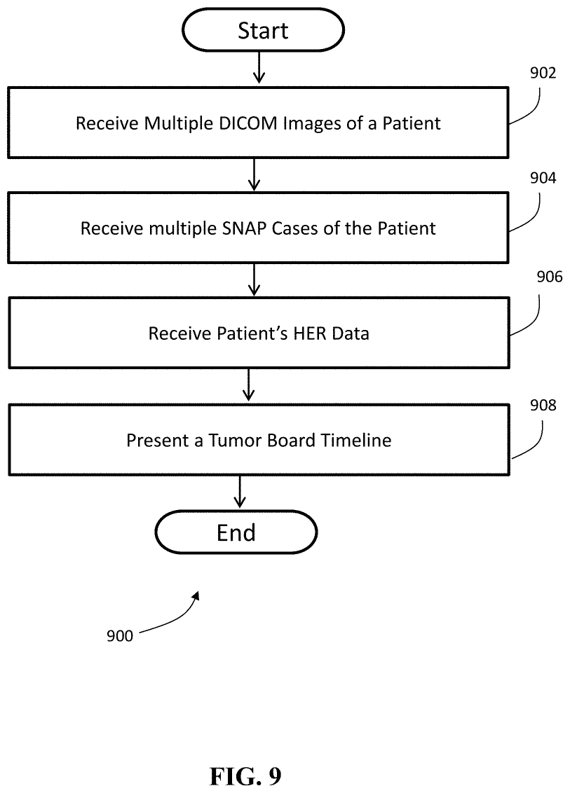

FIG. 9 illustrates an example method for tracking a tumor. At block 902, the tumor tracking computer 102 receives multiple DICOM images of a patient, representative of a patient's tumor over time. At block 904, the tumor tracking computer 102 receives multiple SNAP cases of the patient, representative of the patient's tumor over time. At block 906, the tumor tracking computer 102 receives the patient's electronic health record. At block 908, the tumor tracking computer 102 present a synchronized and integrated tumor board including a timeline for navigating a view of the patient's DICOM, MD6DM models generated based on the SNAP cases, and the electronic health records over time.

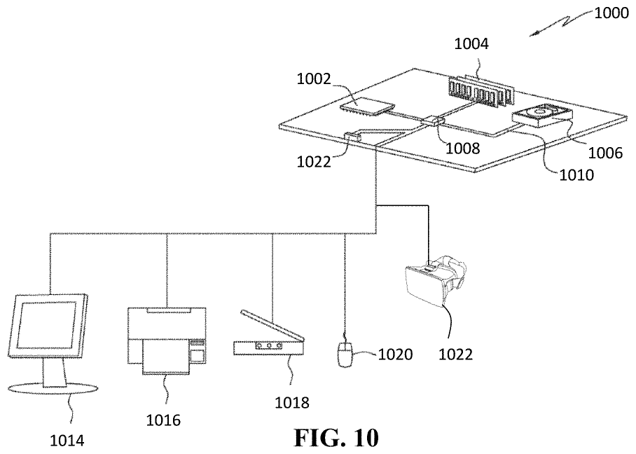

FIG. 10 is a schematic diagram of an example computer 1000 for implementing the example tumor tracking computer 102 of FIG. 1. The example computer 1000 is intended to represent various forms of digital computers, including laptops, desktops, handheld computers, tablet computers, smartphones, servers, and other similar types of computing devices. Computer 1000 includes a processor 1002, memory 1004, a storage device 1006, and a communication port 1008, operably connected by an interface 1010 via a bus 1012.

Processor 1002 processes instructions, via memory 1004, for execution within computer 500. In an example embodiment, multiple processors along with multiple memories may be used.

Memory 1004 may be volatile memory or non-volatile memory. Memory 1004 may be a computer-readable medium, such as a magnetic disk or optical disk. Storage device 1006 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 1004 or storage device 1006.

Computer 1000 can be coupled to one or more input and output devices such as a display 1014, a printer 1016, a scanner 1018, a mouse 1020, and a HMD 1022.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

1. A method of tracking a tumor, comprising:
a tumor tracking computer receiving multiple medical images of a particular patient, representative of the patient's tumor over time;
the tumor tracking computer receiving multiple interactive models of the patient generated using the medical images of the patient, said interactive models being representative of the patient's tumor over time;
the tumor tracking computer generating a multi-dimensional interactive virtual reality view of the tumor in real-time utilizing the multiple interactive models and the multiple medical images;
the tumor tracking computer generating and displaying a synchronized and integrated tumor board including a timeline of the tumor and a viewer displaying the multi-dimensional interactive virtual reality view; and
the tumor tracking computer receiving one or more inputs from a user to navigate the timeline to update the multi-dimensional interactive virtual reality view over time.

2. The method of claim 1, wherein said tumor board includes displaying a view of at least one of said medical images.

3. The method of claim 2, further comprising the steps of:
said tumor tracking computer receiving an input from the user updating at least the one of said medical images; and
said tumor tracking computer updating said multi-dimensional interactive virtual reality view in real-time in response to said input updating the one of said medical images.

4. The method of claim 2, further comprising the steps of:
said tumor tracking computer receiving an input in the tumor tracking computer to display more detailed information about at least one of the medical images; and
said tumor tracking computer displaying more detailed information about the at least one of the medical images in a detailed medical imaging view.

5. The method of claim 1, wherein said inputs from a user to navigate the timeline includes said tumor tracking computer accepting an input for selecting an expanded view that expands said timeline to display more detail in the multi-dimensional interactive virtual reality view for a particular time range.

6. The method of claim 1, further comprising the step of the tumor tracking computer receiving an electronic health record of the patient and wherein said tumor board includes displaying the electronic health records.

7. The method of claim 1, wherein said timeline includes a history of treatments used to treat the tumor displayed in the tumor board.

8. The method of claim 7, wherein said inputs from a user to navigate the timeline includes an input for selecting an expanded view that expands said timeline to display more detail about the treatments in the tumor board for a particular time range.

9. The method of claim 1, wherein said timeline includes a treatment timeline showing treatment(s) used to treat the tumor.

10. The method of claim 9, wherein said timeline also includes a surgery timeline showing surgical procedure(s) used to treat the tumor.

11. The method of claim 9, wherein said treatment(s) include one or more drugs used to treat the tumor.

12. The method of claim 11, said method further comprising the step of said tumor tracking computer accepting an input from the user for displaying detailed information about the one or more drugs used to treat the tumor.

13. The method of claim 1, wherein said timeline includes a surgery timeline showing surgical procedure(s) used to treat the tumor.

14. The method of claim 1, further comprising the step of the tumor tracking computer accepting an input to provide a comparison of sizes of the tumor over time for assessing the effectiveness of the different treatments and surgeries administered over that time based on changes in the tumor.

15. The method of claim 1, where said tumor tracking computer

16. A method of tracking a tumor, comprising:
a tumor tracking computer receiving multiple medical images of a particular patient, representative of the patient's tumor over time;
the tumor tracking computer receiving multiple interactive models of the patient generated using the medical images of the patient, said interactive models being representative of the patient's tumor over time;
the tumor tracking computer receiving an electronic health record of the patient;
the tumor tracking computer generating a multi-dimensional interactive virtual reality view of the tumor in real-time utilizing the multiple interactive models and the multiple medical images;
the tumor tracking computer generating and displaying a synchronized and integrated tumor board, said tumor board including:
displaying a timeline of the tumor including a history of treatment(s) and/or surgery used to treat the tumor,
displaying the electronic health records,
displaying at least the one of said medical images, and
displaying the multi-dimensional interactive virtual reality view; and
the tumor tracking computer receiving one or more inputs from a user to navigate the timeline to update in real-time the display of the medical images viewer and the interactive model viewer over time.

17. The method of claim 16, further comprising the steps of:
receiving an input from the user updating at least the one of said medical images; and
updating said multi-dimensional interactive virtual reality view in real-time in response to said input updating the one of said medical images.

18. The method of claim 16, further comprising the steps of:
receiving an input in the tumor tracking computer to display more detailed information about at least one of the medical images; and
displaying more detailed information about the at least one of the medical images in a detailed medical imaging view.

19. The method of claim 16, wherein said inputs from a user to navigate the timeline includes an input for selecting an expanded view that expands said timeline to display more detail in the multi-dimensional interactive virtual reality view for a particular time range.

20. The method of claim 16, further comprising the step of the tumor tracking computer accepting an input to provide a comparison of sizes of the tumor over time for assessing the effectiveness of the different treatments and surgeries administered over that time based on changes in the tumor

21. A method of tracking a tumor, comprising:
a tumor tracking computer receiving multiple medical images of a particular patient, representative of the patient's tumor over time;
the tumor tracking computer receiving multiple interactive models of the patient generated using the medical images of the patient, said interactive models being representative of the patient's tumor over time;
the tumor tracking computer receiving an electronic health record of the patient;
the tumor tracking computer generating a multi-dimensional interactive virtual reality view of the tumor in real-time utilizing the multiple interactive models and the multiple medical images;
the tumor tracking computer generating and displaying a synchronized and integrated tumor board, said tumor board including:
displaying a timeline of the tumor including a history of treatment(s) and/or surgery used to treat the tumor,
displaying the electronic health records,
displaying at least the one of said medical images, and
displaying the multi-dimensional interactive virtual reality view;
the tumor tracking computer receiving one or more inputs from a user to navigate the timeline to update in real-time the display of the medical images viewer and the interactive model viewer over time, wherein navigating the timeline includes options for:
the tumor tracking computer accepting an input for selecting an expanded view that expands said timeline to display more detail in the multi-dimensional interactive virtual reality view for a particular time range,
the tumor tracking computer accepting an input to display more detailed information about at least one of the medical images, and
the tumor tracking computer accepting an input to provide a comparison of sizes of the tumor over time for assessing the effectiveness of the different treatments and surgeries administered over that time based on changes in the tumor.

21. The method of claim 20, further comprising the steps of:
receiving an input from the user updating at least the one of said medical images; and
updating said multi-dimensional interactive virtual reality view in real-time in response to said input updating the one of said medical images.
